(19) United States
(12) Patent Application Publication
(10) Pub. No.: US 2010/0280192 A1
(43) Pub. Date: Nov. 4, 2010

(54) Fast Curable Epoxy Compositions Containing Imidazole- And 1-(Aminoalkyl) Imidazole-Isocyanate Adducts

(75) Inventors: Maw Lin Foo, Berkeley, CA (US); Gamini Ananda Vedage, Bethlehem, PA (US); Atteye Houssein Abdourazak, Allentown, PA (US)

Correspondence Address: AIR PRODUCTS AND CHEMICALS, INC.;PATENT DEPARTMENT, 7201 HAMILTON BOULEVARD, ALLENTOWN, PA, 181951501, US

(73) Assignee: Air Products and Chemicals, Inc., Allentown, PA (US)

(21) Appl. No.: 12/432,095

(22) Filed: Apr. 29, 2009

(51) Int. Cl. C07D 403/12; C08F 283/10

(52) U.S. Cl. 525/529; 548/313.7

(57) ABSTRACT

Epoxy curing agents comprising imidazole-isocyanate adducts, including 1-(aminoalkyl) imidazole-isocyanate adducts and especially 1-(3-aminopropyl) imidazole-isocyanate adducts, and their use as curing agents in heat curable one-component epoxy resin compositions.

### BACKGROUND OF THE INVENTION

Epoxy based adhesives are used in various applications in automotive, electronics, aerospace and the general industries. They are increasingly replacing conventional bonding systems such as soldering, welding, rivets, nails, screws and bolts because of the benefits they provide over these systems. Some of these benefits include bonding similar and dissimilar substrates without damaging them, better distribution of stress over wide area, better fatigue resistance and noise and vibration resistance. In certain applications in automotive and in electronics, a fast curing (or snap-cure) adhesive system is highly desirable for cycle time reduction and cost savings.

A one-component epoxy based adhesive system is preferred over a two-components system because it eliminates the mixing step, the required time to apply it, and the cooling during storage and shipping associated with the two-components system.

The present invention relates to latent curing agents for epoxy resins including 100% solids epoxy compositions and water-based compositions, especially one-component 100% solids epoxy compositions. “Latent” curing agents are those curatives that in a formulated epoxy system remain inactive under normal ambient conditions but react readily with the epoxy resin at elevated temperatures. “One component” epoxy compositions are typically a blend of an epoxy resin, a curing agent and optionally an accelerator as well as additives and fillers.

### BRIEF SUMMARY OF THE INVENTION

The present invention provides imidazole-isocyanate adducts comprising the reaction products of an imidazole with a polymethylene diisocyanate having 2-8 methylene units or with a polymethylene poly(phenylisocyanate) having an isocyanate functionality of 2.1-3.5 and their use as epoxy curing agents, namely latent curing agents, in heat curable one-component epoxy resin compositions. One-component epoxy resin compositions comprise the contact product of a latent curing agent of this invention, optionally but preferably an accelerator for the curing agent, and an epoxy resin.

In one aspect of the present invention the imidazole-isocyanate adducts are 1-(aminoalkyl) imidazole-isocyanate adducts and in another aspect the imidazole-isocyanate adducts are 1-(3-aminopropyl) imidazole adducts.

The present invention also provides imidazole-isocyanate and 1-(aminoalkyl) imidazole-isocyanate adducts comprising the reaction products of imidazole and 1-(aminoalkyl) imidazole with a polymethylene diisocyanate such as hexamethylene diisocyanate and with polymethylene poly(phenylisocyanate) having an isocyanate functionality of 2.1-3.5 and their use as epoxy curing agents having a fast cure (or snap-cure) at low and high temperature, respectively, as sole, latent curing agents in heat curable one-component epoxy resin compositions.

In one aspect the present invention provides 1-(3-aminopropyl) imidazole-isocyanate adducts comprising the reaction products of 1-(3-aminopropyl)-imidazole with a polymethylene diisocyanate such as hexamethylene diisocyanate and with polymethylene poly(phenylisocyanate) having an isocyanate functionality of 2.1-3.5 as the 1-(aminoalkyl) imidazole-isocyanate adduct curing agents.

In another aspect of the invention the one-component epoxy resin composition comprises a 1-(aminoalkyl) imidazole-isocyanate adduct and an epoxy resin.

In one aspect the invention is directed to imidazole-isocyanate adducts as epoxy curing agents and their use in one-component heat curable epoxy compositions, such as 100% solids compositions and water-based compositions. In another aspect the invention is directed to 1-(aminoalkyl) imidazole-isocyanate adducts as epoxy curing agents and their use in one-component heat curable epoxy compositions, such as 100% solids compositions and water-based compositions.

In all aspects of the invention as stated herein the imidazole-isocyanate adducts may be 1-(aminoalkyl) imidazole-isocyanate adducts and in all aspects the imidazole-isocyanate adducts may be 1-(3-aminopropyl) imidazole-isocyanate adducts. These adducts are prepared by reacting the imidazole with a polymethylene diisocyanate or with a polymethylene poly(phenylisocyanate) having an isocyanate functionality of 2.1-3.5.

In a further aspect of the invention the imidazole-isocyanate adducts are represented by formula A:

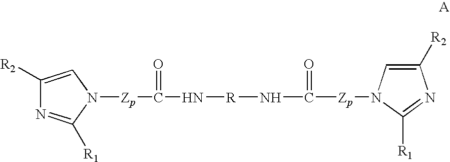

where Z is (CH₂)ₙ—NH, p is 0-1 and n is 3-8, R is (CH₂)ₘ and m is 2-8 or the polyvalent residue of polymethylene poly(phenylisocyanate) having an isocyanate functionality of 2.1-3.5; and R₁ and R₂ independently represent hydrogen, C1-C20 linear or branched alkyl, C6-C10 aryl, alkylaryl or arylalkyl; or C5-C6 cycloaliphatic.

In a yet further aspect of the invention the imidazole-isocyanate adducts are represented by formula B:

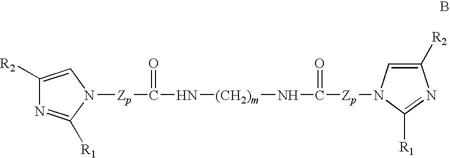

where Z is (CH₂)ₙ—NH, p is 0-1 and n is 3-8, R₁ and R₂ independently represent hydrogen, C1-C20 linear or branched alkyl, C6-C10 aryl, alkylaryl or arylalkyl; or C5-C6 cycloaliphatic; and m is 2-8.

Every aspect and every embodiment of the invention as disclosed herein is meant to be combined with all the other disclosed aspects and embodiments of the invention individually and in all possible combinations thereof.

The term “contact product” is used herein to describe compositions wherein the components are contacted together in any order, in any manner, and for any length of time. For example, the components can be contacted by blending or mixing. Further, contacting of any component can occur in the presence or absence of any other component of the compositions or epoxy formulations described herein. In addition, in contacting the components together two or more of the components may react to form other components.

### BRIEF DESCRIPTION OF THE FIGURES

FIG. 1 is the differential scanning calorimeter cure profile of 1-(3-aminopropyl) imidazole-hexamethylene diisocyanate adduct.

FIG. 2 is the differential scanning calorimeter cure profile of 1-(3-aminopropyl) imidazole-polymethylene poly(phenylisocyanate) adduct.

FIG. 3 is the differential scanning calorimeter cure profile of 1-(3-aminopropyl) imidazole-phenylisocyanate adduct.

FIG. 4 is the differential scanning calorimeter cure profile of 1-(3-aminopropyl) imidazole-cyclohexyl isocyanate adduct.

FIG. 5 is the differential scanning calorimeter cure profile of 1-(3-aminopropyl) imidazole-toluene diisocyanate adduct.

FIG. 6 is the differential scanning calorimeter cure profile of 2-ethyl-4-methyl imidazole-hexamethylene diisocyanate adduct.

FIG. 7 is the differential scanning calorimeter cure profile of imidazole-hexamethylene diisocyanate adduct.

### DETAILED DESCRIPTION OF THE INVENTION

The invention relates to imidazole-isocyanate adducts, including 1-(aminoalkyl) imidazole-isocyanate adducts and especially 1-(3-aminopropyl) imidazole-isocyanate adducts, and their use as curing agents in curing epoxy resin compositions.

Among the various aspects of the invention are the following:
a curing agent for heat cured one-component epoxy compositions.
one-component 100% solids epoxy compositions comprising an imidazole-isocyanate adduct, optionally an accelerator, and an epoxy resin which offer low-temperature and/or high temperature cure and shelf stability.
one-component water based epoxy compositions comprising an imidazole-isocyanate adducts, optionally an accelerator, and an epoxy resin which offer low-temperature and/or high temperature cure and shelf stability.

The fast cure, or snap cure, attribute of the imidazole-isocyanate adducts according to the invention is determined by measuring the cure profile by differential scanning calorimeter (DSC) of the imidazole-isocyanate adducts as epoxy curing agents in an epoxy formulation comprising a polyglycidyl ether of Bisphenol A resin (Epon 828) and 10 phr (wt parts per 100 parts resin) of the curing agent. The resulting mixture is blended thoroughly for 2 minutes using a high sheer cowls blade mixer. Immediately after preparation the mixture is examined by DSC to determine the beginning (Ti) and end (Tf) of the exotherm. The DSC analysis is performed using a 10° C./min ramp heat rate on about a 10 to 15 mg sample of material. For a fast cure according to the invention the value of Tf—Ti is less than about 70° C. in one aspect, less than about 60° C. in another aspect and less than 50° C. in a preferred aspect.

In an aspect of the invention the imidazole-isocyanate adducts are represented by formula A:

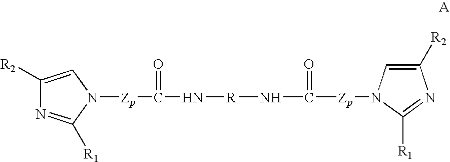

where Z is (CH₂)ₙ—NH, p is 0-1 and n is 3-8, R is (CH₂)ₘ and m is 2-8 or the polyvalent residue of polymethylene poly(phenylisocyanate) having an isocyanate functionality of 2.1-3.5; and R₁ and R₂ independently represent hydrogen, C1-C20 linear or branched alkyl, C6-C10 aryl, alkylaryl or arylalkyl; or C5-C6 cycloaliphatic.

In another aspect of the invention the imidazole-isocyanate adducts are represented by formula B:

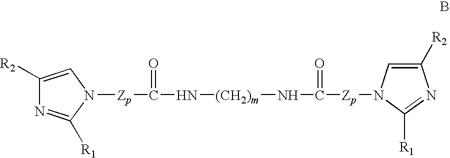

where Z is (CH₂)ₙ—NH, p is 0-1 and n is 3-8, R₁ and R₂ independently represent hydrogen, C1-C20 linear or branched alkyl, C6-C10 aryl, alkylaryl or arylalkyl; or C5-C6 cycloaliphatic; and m is 2-8.

As one aspect of the invention the 1-(aminoalkyl) imidazole-isocyanate adducts for use in epoxy formulations are represented by formula C:

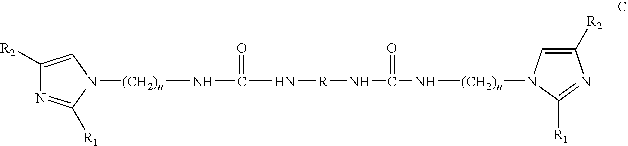

where R is (CH₂)ₘ and m is 2-8 or the divalent residue of polymethylene poly(phenylisocyanate) having an isocyanate functionality of 2.1-3.5; R₁ and R₂ independently represent hydrogen, C1-C20 linear or branched alkyl, C6-C10 aryl, alkylaryl or arylalkyl; or C5-C6 cycloaliphatic; and n is 3-8.

In a further aspect of the invention the 1-(3-aminopropyl) imidazole-isocyanate adducts are represented by formula D:

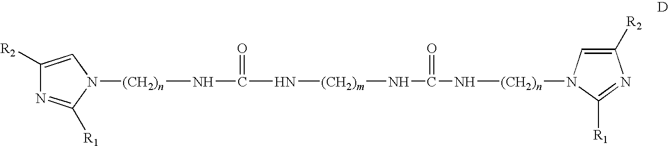

where R₁ and R₂ independently represent hydrogen, C1-C20 linear or branched alkyl, C6-C10 aryl, alkylaryl or arylalkyl; or C5-C6 cycloaliphatic; m is 2-8 and n is 3.

In other aspects of the foregoing aspects and embodiments, independently or in combination m is 3-6 and n is 3-6.

For other aspects of all of the foregoing aspects and embodiments, R represents butanediyl, hexanediyl, octanediyl or the polyvalent residue from polymethylene poly(phenylisocyanate) having an isocyanate functionality of 2.1-3.5; and R₁ and R₂ substituents are selected individually or in any combination from the group consisting of representing hydrogen, C1-C4 linear or branched alkyl, C6-C8 aryl, alkylaryl or arylalkyl, especially hydrogen, methyl and ethyl.

Yet in other aspects of each of the foregoing aspects and embodiments, R represents hexanediyl or the polyvalent residue from polymethylene poly(phenylisocyanate) having an isocyanate functionality of 2.1-3.5; and R₁ and R₂ substituents are selected individually or in any combination from the group consisting of hydrogen, C1-C4 linear or branched alkyl, C6-C8 aryl, alkylaryl or arylalkyl, especially hydrogen, methyl and ethyl.

Suitable alkyl groups include, for example, methyl, ethyl, n- and isopropyl, n-, iso-, sec- and tert-butyl, 2-ethyhexyl, octyl, decyl, dodecyl and heptadecyl. Suitable aryl groups include phenyl and naphthyl. Suitable arylalkyl group include benzyl and phenyl ethyl. Suitable alkylaryl groups include tolyl, xylyl and ethylphenyl. Suitable C5-C6 cycloaliphatic groups include cyclopentyl and cyclohexyl.

In other embodiments of the various aspects of the invention, R₁ and R₂ independently represent hydrogen, methyl, ethyl, linear or branched propyl or butyl, benzyl, phenylethyl, cyclopentyl and cyclohexyl. In other embodiments of the various aspects of the invention, R₁ and R₂ independently represent hydrogen, methyl, or ethyl. In other embodiments of the various aspects of the invention, R₁ and R₂ represent hydrogen.

In one aspect of R in the above formulas, R is a C3-C8 alkanediyl.

In another aspect of R in the above formulas, R is the polyvalent residue of polymethylene poly(phenylisocyanate) having an isocyanate functionality of 2.1-3.5, the isocyanate functionalities having reacted with the nitrogen atom of the imidazoles to afford urea functionalities.

Every aspect and every embodiment of the invention as disclosed herein is meant to be combined with all the other disclosed aspects and embodiments of the invention individually and in all possible combinations thereof. Such combinations would be apparent to a skilled worker in the art from the present description of the invention.

The imidazole-isocyanate adducts including the 1-(aminoalkyl) imidazole-isocyanate adducts and especially the 1-(3-aminopropyl) imidazole-isocyanate adducts, can be prepared by reactions well known to a chemist and are reported in the literature such as U.S. Pat. No. 4,797,455 or by reacting an appropriate imidazole with the appropriate polyisocyanate, i.e., polymethylene diisocyanate or polymethylene poly(phenylisocyanate), in a polar organic solvent such as acetonitrile. The imidazole and polyisocyanate are reacted in an equivalents ratio of imidazole to polyisocyanate of at least 0.4:1, preferably 0.5 to 1.5:1, especially 1:1.

Imidazoles and isocyanates suitable for use in making the adducts are commercially available. Imidazoles are available from Sigma-Aldrich, Shikoku Chemical and BASF. Isocyanates are available from Sigma-Aldrich, Bayer and Huntsman.

In an aspect of the invention suitable imidazole-isocyanate adducts include adducts of tetramethylene diisocyanate, pentamethylene diisocyanate, hexamethylene diisocyanate, octamethylene diisocyanate, and polymethylene poly(phenylisocyanate) with imidazoles such as imidazole, 2-ethyl imidazole, 2-methyl imidazole, 2-propyl imidazole, 2-undecyl imidazole, 2-phenyl imidazole, 2-isopropyl imidazole, 2-ethyl-4-methyl imidazole, 2-phenyl-4-methyl imidazole and 1-(aminoalkyl) imidazoles such as 2-methyl-1-(3-aminopropyl) imidazole, 2-ethyl-1-(3-aminopropyl) imidazole, 2-phenyl-1-(3-aminopropyl) imidazole, 2-ethyl-4-methyl-1-(3-aminopropyl) imidazole. The preferred imidazole-isocyanate adducts include imidazole and 2-ethyl-4-methyl imidazole-hexamethylene diisocyanate and polymethylene poly(phenylisocyanate) adducts, 1-(3-aminopropyl) imidazole-hexamethylene diisocyanate adduct and 1-(3-aminopropyl) imidazole-polymethylene poly(phenylisocyanate) adduct.

The imidazole-isocyanate adducts can be used as epoxy curing agents in one-component epoxy compositions such as adhesives, decorative and protective coatings including powder coatings, filament winding, printed circuit board and other epoxy applications. Typically, 0.5 to 10 parts by weight (pbw) imidazole-isocyanate adduct are used in the epoxy composition per 100 pbw epoxy resin, preferably 2 to 6 pbw of imidazole-isocyanate adduct per 100 pbw epoxy resin.

The imidazole-isocyanate adduct epoxy curing agent is combined with an epoxy resin which is a polyepoxy compound containing more than one 1,2-epoxy groups per molecule. Such epoxides are well known in the epoxy art and are described in Y. Tanaka, “Synthesis and Characteristics of Epoxides”, in C. A. May, ed., Epoxy Resins Chemistry and Technology (Marcel Dekker, 1988). Examples include those epoxides disclosed in U.S. Pat. No. 5,599,855 (Col 5/6 to 6/20), which is incorporated by reference. The preferred polyepoxy compounds are the diglycidyl ethers of bisphenol-A, the advanced diglycidyl ethers of bisphenol-A, the diglycidyl ethers of bisphenol-F, and the epoxy novolac resins. Both liquid epoxy resins and solid epoxy resins are suitably used in the one component epoxy compositions. Powder coating compositions would comprise a solid epoxy resin and an imidazole-isocyanate adduct and, optionally, an accelerator.

Epoxy compositions prepared from imidazole-isocyanate adducts can be formulated with a wide variety of ingredients well known to those skilled in the art of coating formulation, including solvents, fillers, pigments, pigment dispersing agents, rheology modifiers, thixotropes, flow and leveling aids, and defoamers.

While one component epoxy compositions comprising 1 to 90 wt % organic solvents, or 100 wt % solids epoxy compositions, or water-based, i.e., aqueous, epoxy compositions containing 20 to 80 wt % solids can be used, it is preferred the epoxy composition be 100 wt % solids.

The epoxy compositions of this invention can be applied as coatings by any number of techniques including spray, brush, roller, paint mitt, and the like. Numerous substrates are suitable for application of coatings of this invention with proper surface preparation, as is well understood in the art. Such substrates include but are not limited to many types of metal, particularly steel and aluminum, as well as concrete.

One component epoxy compositions of this invention can be cured at elevated temperatures ranging from about 80° C. to about 240° C., with cure temperatures of 120° C. to 160° C. preferred.

### Examples A-G

### Example A

Into a three neck flask on a heating mantle fitted a with condenser, a dropping funnel and thermocouple was charge 50 g (0.4 mole) of 1-(3-aminopropyl) imidazole and 250 ml of acetonitrile and heated to 70-80° C. To the stirred solution was added drop wise 33.6 g (0.2 mole) of hexamethylene diisocyanate dissolved in 50 ml of acetonitrile. White precipitate formed. After the addition the mixture was stirred at 70-80° C. for an hour then cooled to ambient temperature. The white solid was filtered and washed with acetonitrile.

### Example B

1-(3-aminopropyl) imidazole (63 g; 0.5 mole) and polymethylene polyphenylisocyanate (82.9 g; 0.3 mole) were reacted following the procedure of Example A. The 1-(3-aminopropyl) imidazole-polymethylene polyphenylisocyanate adduct was obtained as a solid product.

### Example C

1-(3-aminopropyl) imidazole (62.59 g; 0.5 mole) and phenylisocyanate (59.5 g; 0.5 mole) were reacted following the procedure of Example A. The 1-(3-aminopropyl) imidazole-phenylisocyanate adduct was obtained as a solid product.

### Example D

1-(3-aminopropyl) imidazole (30 g; 0.24 mole) and cyclohexylisocyanate (30 g; 0.24 mole) were reacted following the procedure of Example A. The 1-(3-aminopropyl) imidazole-cyclohexylisocyanate adduct was obtained as a viscous clear yellow product.

### Example E

1-(3-aminopropyl) imidazole (63 g; 0.5 mole) and toluene diisocyanate (46.1 g; 0.26 mole) were reacted following the procedure of Example A. The 1-(3-aminopropyl) imidazole-toluene diisocyanate adduct was obtained as a solid product.

### Example F

2-Ethyl-4-methyl imidazole (22 g; 0.2 mole) and hexamethylene diisocyanate (16.8 g; 0.1 mole) were reacted following the procedure of Example A. The 2-ethyl-4-methyl imidazole-hexamethylene diisocyanate adduct was obtained as a solid product.

### Example G

Imidazole (13.2 g; 0.2 mole) and hexamethylene diisocyanate (16.8 g; 0.1 mole) were reacted following the procedure of Example A. The imidazole-hexamethylene diisocyanate adduct was obtained as a solid product.

The imidazole-isocyanate adducts of Examples A-G were screened by differential scanning calorimeter (DSC) for their cure profile as epoxy curing agents. The epoxy formulation comprised polyglycidyl ether of Bisphenol A resin (Epon 828) and 10 phr (wt parts per 100 parts resin) of the curing agent. The resulting mixtures were blended thoroughly for 2 minutes using a high sheer cowls blade mixer. Immediately after preparation the mixtures were examined by DSC to determine the onset temperature (To), the heat of reaction (ΔH), the maximum exotherm (Tmax), the beginning (Ti) and end (Tf) of the exotherm. The DSC analysis was performed using a 10° C./min ramp heat rate on about a 10 to 15 mg sample of material. The resulting data is shown in Table 1.

TABLE 1

| | | | | | T_{f} − Tᵢ | ΔH |
|---|---|---|---|---|---|---|
| Example | T_{O} (° C.) | Tₘₐₓ (° C.) | Tᵢ (° C.) | T_{f} (° C.) | (° C.) | (J/g) |
| A | 129 | 140 | 121 | 156 | 35 | 419 |
| B | 181 | 190 | 162 | 220 | 58 | 368 |
| C | 110 | 127 | 97 | 187 | 90 | 352 |
| D | 84 | 106 | 49 | 182 | 133 | 288 |
| E | 117 | 138 | 96 | 229 | 133 | 412 |
| F | 127 | 135 | 100 | 169 | 69 | 485 |
| G | 124 | 137 | 98 | 165 | 67 | 513 |

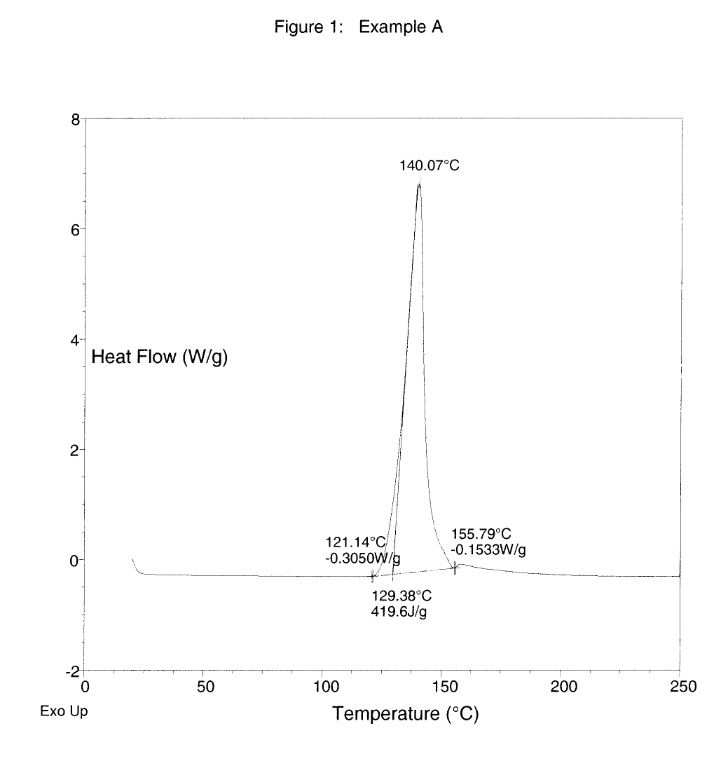

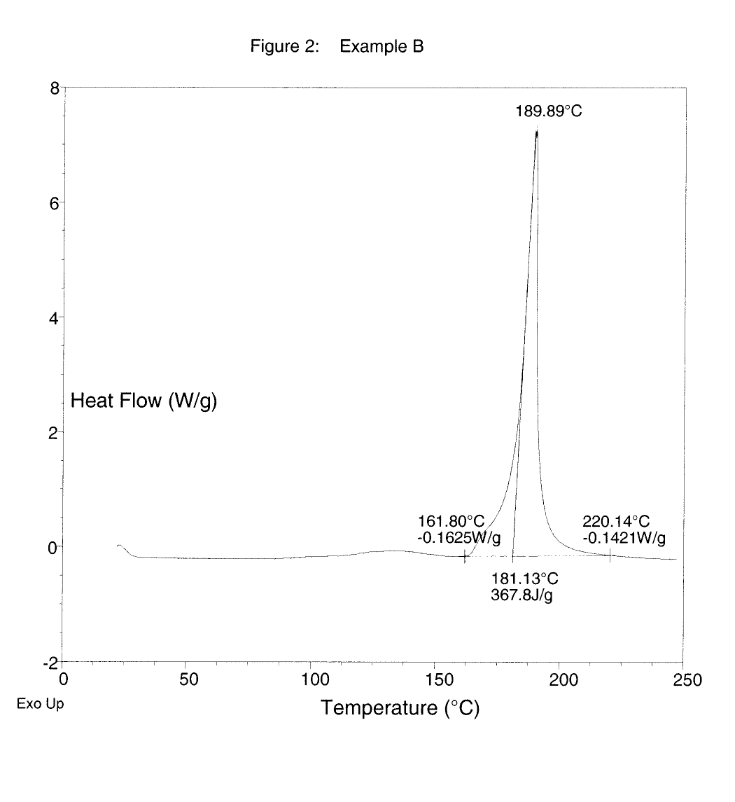

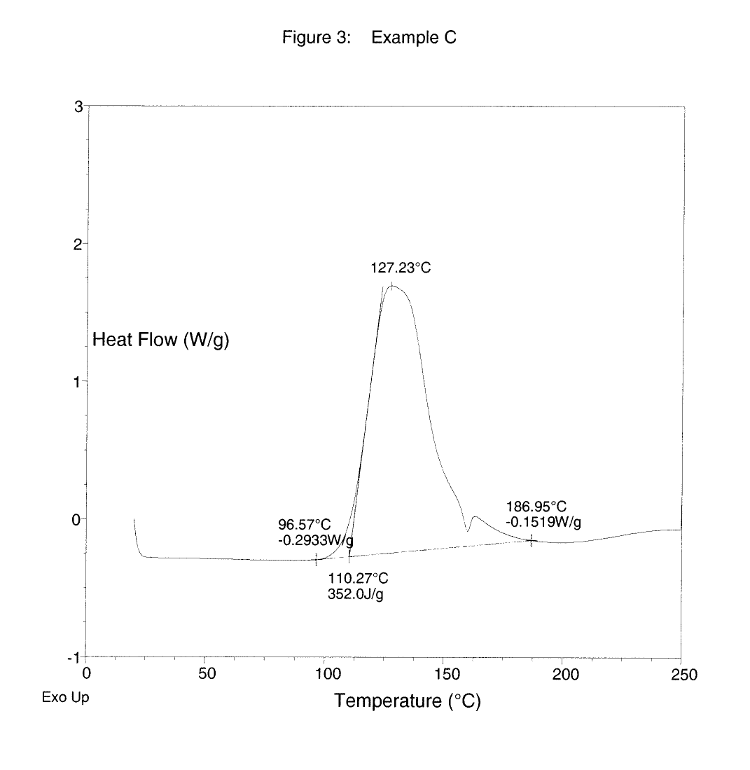

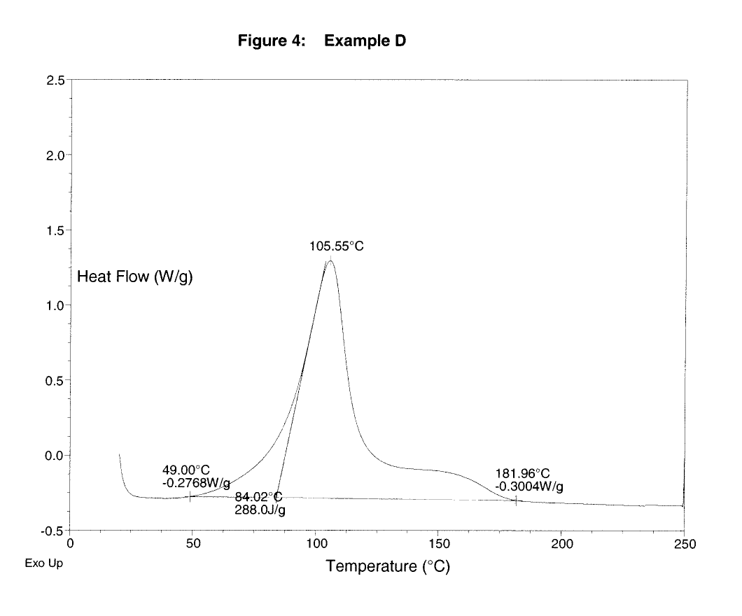

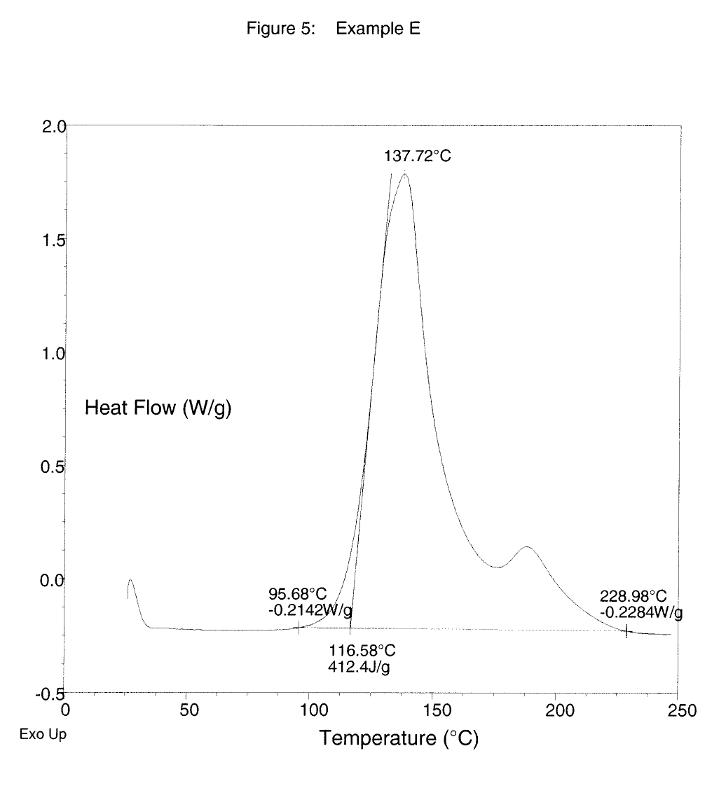

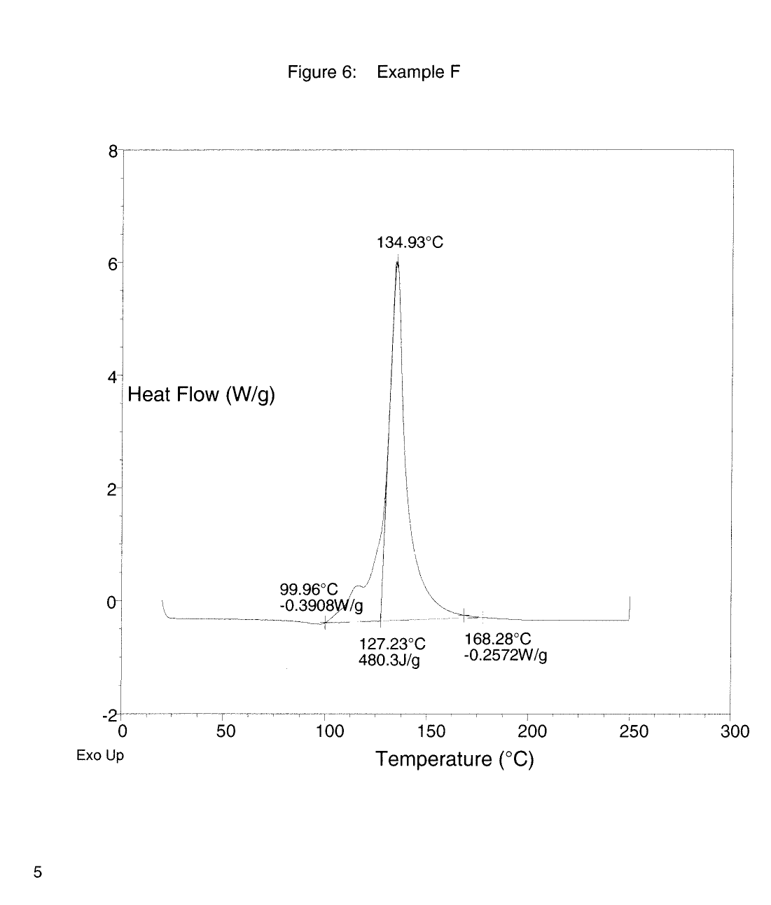

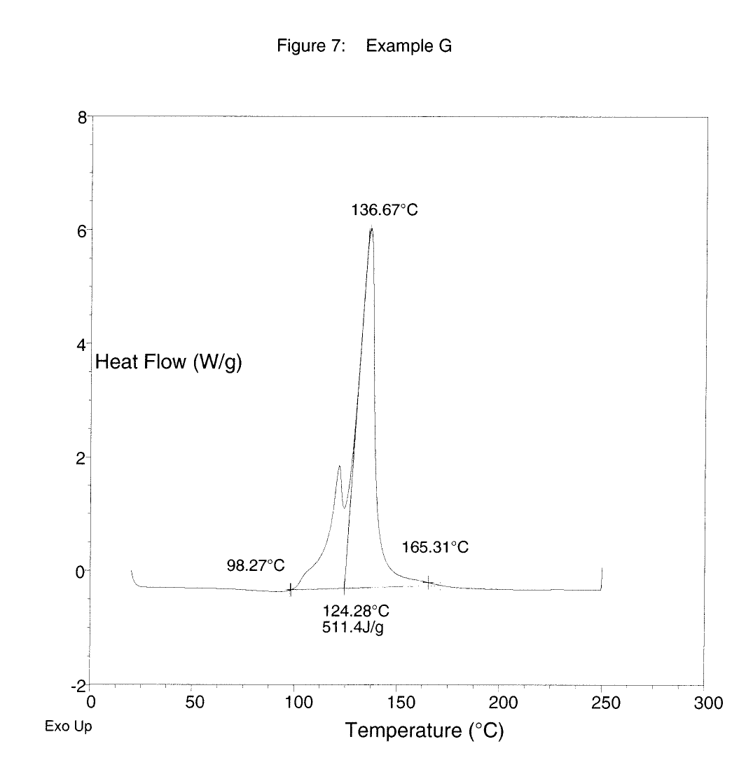

The 1-(3-aminopropyl) imidazole, the 2-ethyl-4-methyl-imidazole and the imidazole adducts obtained from hexamethylene diisocyanate were found to have a fast cure, or snap-cure, profile at low temperature (140° C.) and the 1-(3-aminopropyl) imidazole-polymethylene poly(phenylisocyanate) adduct had a fast cure, or snap cure, profile at high temperature (190° C.), as shown by their narrow peaks in FIGS. 1, 2, 6 and 7 which translate into a small difference between the start and end of the exotherm (Tf-Ti). All the other adducts exhibited a broad cure profile as shown in FIGS. 3-5.

A fast cure, or snap-cure, profile is highly desirable in high volume applications where cycle time reduction and cost savings are important.

I claim:

1. An epoxy curing agent composition comprising an imidazole-isocyanate adduct which comprises the reaction product of an imidazole and a polymethylene diisocyanate having 2-8 methylene units or a polymethylene poly(phenylisocyanate) having an isocyanate functionality of 2.1-3.5.

2. The epoxy curing agent of claim 1 in which the imidazole-isocyanate adduct is represented by formula A: 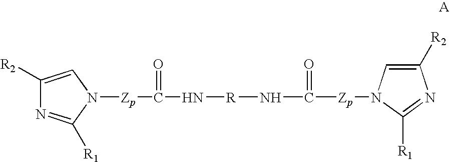
where Z is (CH₂)ₙ—NH, p is 0-1 and n is 3-8, R is (CH₂)ₘ and m is 2-8 or the polyvalent residue of polymethylene poly(phenylisocyanate) having an isocyanate functionality of 2.1-3.5; and R₁ and R₂ independently represent hydrogen, C1-C20 linear or branched alkyl, C6-C10 aryl, alkylaryl or arylalkyl; or C5-C6 cycloaliphatic.

3. The epoxy curing agent of claim 1 in which the imidazole-isocyanate adduct is represented by formula B: 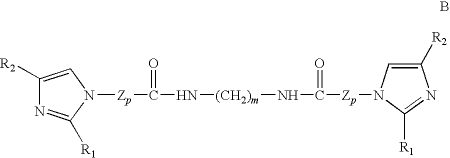
where Z is (CH₂)ₙ—NH, p is 0-1 and n is 3-8, R₁ and R₂ independently represent hydrogen, C1-C20 linear or branched alkyl, C6-C10 aryl, alkylaryl or arylalkyl; or C5-C6 cycloaliphatic; and m is 2-8.

4. A curable epoxy composition comprising the contact product of an epoxy resin and an epoxy curing agent comprising an imidazole-isocyanate adduct which is the reaction product of an imidazole and a polymethylene diisocyanate having 2-8 methylene units or a polymethylene poly(phenylisocyanate) having an isocyanate functionality of 2.1-3.5.

5. The curable epoxy composition of claim 4 in which the imidazole-isocyanate adduct is represented by formula A: 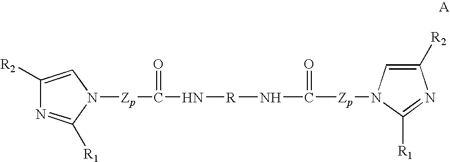
where Z is (CH₂)ₙ—NH, p is 0-1 and n is 3-8, R is (CH₂)ₘ and m is 2-8 or the polyvalent residue of polymethylene poly(phenylisocyanate) having an isocyanate functionality of 2.1-3.5; and R₁ and R₂ independently represent hydrogen, C1-C20 linear or branched alkyl, C6-C10 aryl, alkylaryl or arylalkyl; or C5-C6 cycloaliphatic.

6. The curable epoxy composition of claim 4 in which the imidazole-isocyanate adduct is represented by formula B: 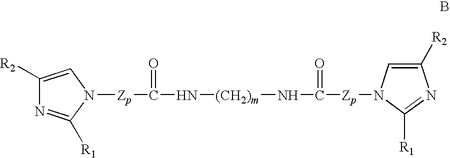
where Z is (CH₂)ₙ—NH, p is 0-1 and n is 3-8, R₁ and R₂ independently represent hydrogen, C1-C20 linear or branched alkyl, C6-C10 aryl, alkylaryl or arylalkyl; or C5-C6 cycloaliphatic; and m is 2-8.

7. The curable epoxy composition of claim 4 in which the imidazole-isocyanate adduct is a 1-(aminoalkyl) imidazole-isocyanate adduct represented by formula C: 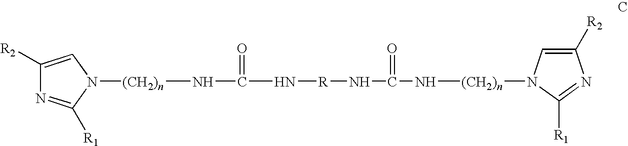
where R is (CH₂)ₘ and m is 2-8 or the polyvalent residue of a polymethylene poly(phenylisocyanate) having an isocyanate functionality of 2.1-3.5; R₁ and R₂ independently represent hydrogen, C1-C20 linear or branched alkyl, C6-C10 aryl, alkylaryl or arylalkyl; or C5-C6 cycloaliphatic; and n is 3-8.

8. The curable epoxy composition of claim 4 in which the imidazole-isocyanate adduct is a 1-(aminoalkyl) imidazole-isocyanate adduct represented by formula D: 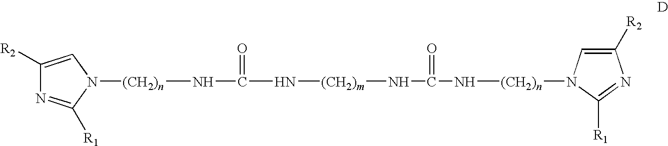
where R₁ and R₂ independently represent hydrogen, C1-C20 linear or branched alkyl, C6-C10 aryl, alkylaryl or arylalkyl; or C5-C6 cycloaliphatic; m is 2-8 and n is 3.

9. The epoxy composition of claim 5 in which R is (CH₂)ₘ, m is 3-6 and n is 3-6.

10. The epoxy composition of claim 5 in which R is the polyvalent residue of a polymethylene poly(phenylisocyanate) having an isocyanate functionality of 2.1-3.5.

11. The epoxy composition of claim 4 which is a 100% solids epoxy composition.

12. The epoxy composition of claim 4 which is an aqueous epoxy composition of 20 to 80 wt % solids.

13. The epoxy composition of claim 9 in which m is 6.

14. The epoxy composition of claim 9 in which n is 3.

15. A heat curable one-component epoxy composition comprising the contact product of an epoxy resin, a latent heat activated curing agent and optionally an accelerator for the latent curing agent, the latent curing agent comprising a 1-(aminoalkyl) imidazole-isocyanate adduct represented by the formula A: 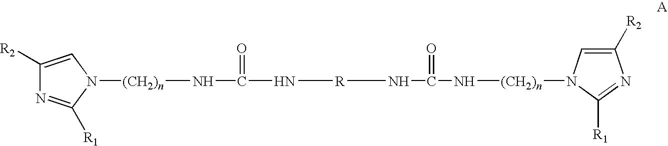
where R is (CH₂)ₘ and m is 3-6 or the polyvalent residue of a polymethylene poly(phenylisocyanate) having an isocyanate functionality of 2.1-3.5; R₁ and R₂ independently represent hydrogen, C1-C4 linear or branched alkyl, C6-C8 aryl, alkylaryl or arylalkyl; and n is 3-6.

16. The epoxy composition of claim 15 in which R₁ and R₂ independently represent hydrogen, methyl or ethyl.

17. The epoxy composition of claim 15 in which m is 6.

18. The epoxy composition of claim 15 in which n is 3.

19. The epoxy composition of claim 15 which is a 100% solids epoxy composition.

20. The epoxy composition of claim 15 which is an aqueous epoxy composition of 20 to 80 wt % solids.

21. The epoxy composition of claim 19 in which R is (CH₂)ₘ.

22. The epoxy composition of claim 15 in which R is the polyvalent residue of a polymethylene poly(phenylisocyanate) having an isocyanate functionality of 2.1-3.5.

23. A method for providing a fast cure profile in an epoxy composition which comprises heating and curing an epoxy composition of claim 4 in which the difference between the beginning (Ti) and end (Tf) of the exotherm is less than 70° C. as determined by DSC analysis.

24. The method of claim 23 in which the difference between the beginning (Ti) and end (Tf) of the exotherm is less than 60° C. as determined by DSC analysis.

25. A method for providing a fast cure profile in an epoxy composition which comprises heating and curing an epoxy composition of claim 15 in which the difference between the beginning (Ti) and end (Tf) of the exotherm is less than 70° C. as determined by DSC analysis.
